(19) United States
(12) Patent Application Publication
(10) Pub. No.: US 2004/0076641 A1
(43) Pub. Date: Apr. 22, 2004

(54) Herbal dietary supplement

(76) Inventor: Timothy Kershenstine JR., Metairie, LA (US)

Correspondence Address: THOMAS S. KEATY, KEATY PROFESSIONAL LAW CORP., 2140 WORLD TRADE CENTER, NO. 2 CANAL STREET, NEW ORLEANS, LA, 70130, US

(21) Appl. No.: 10/684,664

(22) Filed: Oct. 14, 2003

Related U.S. Application Data
(60) Provisional application No. 60/418,531, filed on Oct. 15, 2002.

(51) Int. Cl. A61K 35/84; A61K 35/78

(52) U.S. Cl. 424/195.15; 424/728; 424/764

(57) ABSTRACT

A dietary supplement for ingestion by humans is formulated to reduce blood pressure level, increase energy and promote general wellbeing. The supplement contains, in a tablet or capsule form, as active ingredients Spica Prunella flos, Chrysanthemi flos, Lonicera Japonica, Radix Notoginseng, Cleistocalyx Operculatis, Lentinus Edodes, and Sophora Japonica.

### CROSS-REFERENCE TO RELATED APPLICATIONS

This application is based on and claims the priority benefits of my provisional application Serial No. 60/418,531 filed on Oct. 15, 2002 entitled “Balance3 Dietary Supplement,” the full disclosure of which is incorporated by reference herein.

### FIELD OF THE INVENTION

This invention relates to herbal dietary supplement compositions useful for improving or maintaining normal blood pressure, energy level and general physical wellness.

### BACKGROUND OF THE INVENTION

According to the U.S. Public Health Service, about 50% Americans over the age of 65 suffer from the effects of elevated blood pressure or hypertension. Men appear to have high blood pressure more than women, although post-menopausal women have higher risk levels of the disease, which approaches that for men. Many individuals do not have any symptoms of the hypertension until severe problems cause them to take notice. An increasing number of Americans seem to suffer from this condition, which in some cases causes fatal outcome. Often people with hypertension are eating a poor diet with not enough fruits and vegetables. Doctors generally recommend that patients restrict salt in their diets. Nutritionists suggest eating products containing omega fatty acids, such as fatty fish and nuts. Health food enthusiasts recommend drinking 6-8 glasses of water a day, eating lots of raw fruits and vegetables (including garlic and onions), drinking fresh-made juices, avoiding coffee, alcohol, processed foods white sugar, white flour, red meat and animal fat, etc.

While these recommendations are sound and can be successfully followed by persons with low to normal blood pressure, there continues to be a need for some type of a dietary supplement, which is based on natural products and can be taken to lower elevated pressure without resorting to prescription medications.

The present invention contemplates provision of such a dietary supplement, which is based on natural plants and herbs and which can be taken in a convenient pill form.

### SUMMARY OF THE INVENTION

It is, therefore, an object of the present invention to provide an herbal dietary supplement that can be used for decreasing the effects of high blood pressure.

It is another object of the present invention to provide an herbal supplement that may be used for increasing energy levels and facilitating general wellness in humans.

These and other objects of the present invention are achieved through a provision of a stable dietary supplement composition, which contains as active ingredients Spica Prunella (All-heal flower), Chrysanthemi flos (Chrysanthemum flower), Lonicera japonica (Honeysuckle flower), Radix notoginseng (Notoginseng root), Cleistocalyx operculatus (Water Fairy flower), Leninus edodes Sing. (Shiitake mushroom), and Sophora Japonica (Pagoda tree flower). The composition is administered daily in the form of 1-3 tablets or capsules per day.

All ingredients are natural herbs or plant parts that are processed by extracting or granulating, as necessary to form a homogenized mixture, which is then compressed into a tablet or capsule.

### DETAILED DESCRIPTION OF THE PREFERRED EMBODIMENT

The dietary supplement product of the present invention is based on natural herbal ingredients that are combined in a formula that has improved effects in lowering blood pressure. In addition, the dietary supplement of the present invention promotes general wellness and increases energy level in human subjects. It a preferred embodiment, the composition of herbal ingredients comprises Spica Prunella (All-heal flower), Chrysanthemi flos (Chrysanthemum flower), Lonicera japonica (Honeysuckle flower), Radix notoginseng (Notoginseng root), Cleistocalyx operculatus (Water Fairy flower), Leninus edodes Sing.(Shiitake mushroom), and Sophora Japonica (Pagoda tree flower).

Spica Prunella, or Prunella vulgaris L. is a common plant found in woods and fields in many locations. In herbal medicine, it was primarily known as an astringent for inward and outward use. It was also known as a remedy alleviating pains in the throat, fevers and accelerating wound healing. A high content of rosmarinic acid, immunomodulation effects of the polysaccharide prunelline and antiviral activity of some constituents made the plant interesting from the viewpoint of therapeutical applications.

Chrysanthemum, or as commonly known feverfew, has been used in the Oriental medicine for Iiproving visual acuity, for reducing fever, dizziness and headache. It is also said useful in decreasing the symptoms of conjunctival congestion with swelling pain. Traditionally, the herbal medicine uses the flower-head of a familiar garden flower.

Lonicera Japonica Flos, or Honeysuckle flower is known as a broad-spectrum antibiotic, anti-inflammatory agent, and antipyretic. The Lonicera contains chlorogenic acid, isochlorogenic acid and luteolin. As an aqueous distillate (1:1280) it is reported to promote white cell phagocytosis and enhance lymphocyte-blastogenesis rate.

Notoginseng Radix, or Pseudoginseng, ahs been used in Chinese medicine to reduce bruising, swelling, and pain. Traditional Chinese medicine regards this plant as a superb blood tonic and blood cleanser when cooked. It is a powerful blood vitalizing agent when consumed raw, and is believed by the Chinese to protect the heart and vascular system. It can be used externally to stop bleeding. It is widely used in Chinese herbalism internally as a tonic after traumatic injury.

Cleistocalyx operculatus, or Water Fairy, is an herb commonly used as an ingredient for tonic drinks in Southern China. It is believed to assist heart contraction, slowing rapid heartbeat. Some studies suggest that the plant provides positive inotropic and negative chronotropic actions on heart.

Shiitake mushroom, also called Japanese mushroom, is reported to be beneficial for preventing high blood pressure and heart disease, for controlling cholesterol level, building resistance against viruses, and fighting diseases such as cancers. Shiitake Mushrooms contain a polysaccharide called “lentinan” that has been shown to slow the growth of cancerous tumors in animals. Studies suggest that lentinan may work by enhancing the immune system's ability to fight against infection. Shiitake Mushrooms is used as a cancer-fighting agent in Japan and China. Shiitake also reported to lower cholesterol, which helps prevent heart disease, and act as an anti-viral agent and as an anticoagulant helping the body to excrete excess cholesterol.

Sophora japonica flos (Pagoda tree flower buds) have been used in Chinese medicine and herbal teas. It is believed that Sophora Japonica helps regulate blood fat and improves microcirculation. In traditional Chinese medicine, it is used in a health drink designed to treat sufferers of hyperlipidemia, hypertension, obesity and arteriosclerosis.

Although the mechanism of the dietary supplement of the present invention is not clear, it is believed to have beneficial effect on hypertension and cholesterol level of humans. It helps to dilate the blood vessels and also helps with coronary blood flow tby increasing blood circulation.

To prepare the dietary supplement composition of this invention, the following method has been followed. Each of the seven ingredients of raw materials is individually sterilized. Chrysanthemum flower, Notoginseng root, honeysuckle flower, and Pagoda tree flower are ultra micro-comminuted by an airflow grinder. Water Fairy flower and Shiitake mushrooms are distilled, extracted and then volatilized. All-Heal Flower is processed in a CO2 Super-critical extractive equipment to form an extract of the plant.

The ground Chrysanthemum flower, Notoginseng root, Honeysuckle flower, and Pagoda tree flower are then deposited into a granulator together with a extract of All-Heal flower. The extract of Water Fairy flower and Shiitake mushrooms is sprayed on the granules and all ingredients are deposited into a three-dimensional mixer. The active ingredients are mixed to form a cream and admixed with added non-active auxiliary materials, such as binding agents containing cellulose, magnesium stearate and dextrose.

After mixing for a pre-determined period of time, the mixture is transferred to a tablet press and pressed into pills. The finished product has 15 grams of compliments, which comprise 1.5 g magnesium stearate, 5.5 g cellulose, 8.0 g dextrose. The active ingredients comprise 79 g of the finished product, which us compressed into 1000 tablets, each weighing 0.079 g±7.5%. The final product has yellowish brown color.

Alternatively, the product may be formed as capsules following the steps of: cleaning the ingredients, ultra micro-comminuting the lyophilize Chrysanthemum flower, Notoginseng root, Honeysuckle flower, Pagoda Tree flower, All-Heal Flower, Water Fairy flower and Shiitake mushrooms. The resultant ingredients are deposited into a granulator and processed. 80 g of the granules are then deposited into a capsule press, pressing into capsules, which comprise 20 g of compliments. The compliments in the capsule formulation comprise rice flour. 100 g of the resultant mixture is compressed into 1000 capsules, each weighing 0.10 g±7.5%. The finished capsules are brown in color.

It is envisioned that other forms of the dietary supplement of the present invention may be made, such as for instance, quick-dissolve tablets, liquid capsules, infusions, liquid extracts, and the like.

To make 800 g of active ingredients in dry form, the preferred embodiment of the dietary supplement of the present invention comprises equal amounts of about 150 g (18.75% wt) each of All-Heal flower and Chrysanthemum flower, about 120 g (15% wt) Honeysuckle flower, equal amounts of about 100 g (12.5% wt) each of Notoginseng root and Water Fairy flower, equal amounts of about 90 g (11.25% wt) each of Shiutake mushrooms and Pagoda Tree flower.

Provisional clinical trials indicate that subjects ingested the tablets or capsules without apparent side effect to help with the symptoms of hypertension, decreased energy level, and insomnia. The subjects were taking 1-3 tablets daily.

Many changes and modifications can be made in the composition of the present invention without departing from the spirit thereof. I, therefore, pray that my rights to the present invention be limited only by the scope of the appended claims.

I claim:

1. A stable dietary supplement composition containing herbal ingredients selected from a group consisting of Spica Prunella flos, Chrysanthemi flos, Lonicera Japonica, Radix Notoginseng, Cleistocalyx Operculatis, Lentinus Edodes, and Sophora Japonica.

2. The dietary supplement composition of claim 1, wherein Cleistocalyx Operculatis and Lentinus Edodes are accessed by distilling and extracting.

3. The dietary supplement composition of claim 1, wherein Spica Prunella is accessed by extracting.

4. The dietary supplement composition of claim 1, wherein the active ingredients comprise about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica.

5. The dietary supplement composition of claim 1, wherein said supplement is a tablet with binding agents containing at least cellulose, magnesium stearate and dextrose.

6. The dietary supplement composition of claim 1, wherein said supplement is a capsule with a binding agent containing rice flour.

7. The dietary supplement composition of claim 1, wherein the composition comprises a tablet or capsule.

8. A stable dietary supplement composition for decreasing and maintaining normal blood pressure in humans comprising as active ingredients about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica.

9. The dietary supplement composition of claim 8, wherein said supplement is a tablet with binding agents containing at least cellulose, magnesium stearate and dextrose.

10. The dietary supplement composition of claim 8, wherein said supplement is a capsule with a binding agent containing rice flour.

11. A stable dietary supplement composition for lowering high blood pressure, the composition containing an effective amount of active ingredients selected from a group consisting of Spica Prunella flos, Chrysanthemi flos, Lonicera Japonica, Radix Notoginseng, Cleistocalyx Operculatis, Lentinus Edodes, and Sophora Japonica, and where said composition is a tablet or capsule containing binding agents.

12. The composition of claim 11, wherein said composition comprises about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica.

13. A method of reducing blood pressure levels in humans, comprising the daily administration to a human of a composition containing as active ingredients Spica Prunella flos, Chrysanthemi flos, Lonicera Japonica, Radix Notoginseng, Cleistocalyx Operculatis, Lentinus Edodes, and Sophora Japonica.

14. The method of claim 13, wherein said composition comprises about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica.

15. A dietary supplement in tablet form, comprising about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica.

16. The supplement of claim 15, further comprising binding agents containing cellulose, magnesium stearate and dextrose.

17. A dietary supplement in capsule form, comprising about 18.75% wt each Spica Prunella flos and Chrysanthemi flos, about 15% wt of Lonicera Japonica, about 12.5% wt each of Radix Notoginseng and Cleistocalyx Operculatis, and about 11.25% wt each of Lentinus Edodes and Sophora Japonica and further comprising rice flour as a binding agent.
